(19) United States
(12) Patent Application Publication
(10) Pub. No.: US 2005/0033151 A1
(43) Pub. Date: Feb. 10, 2005

(54) Combined magnetic resonance data acquisition of multi-contrast images using variable acquisition parameters and k-space data sharing

(76) Inventors: Ed X Wu, New York, NY (US); Ralf Mekle, New York, NY (US); Robert Delapaz, Dobbs Ferry, NY (US)

Correspondence Address: BAKER & BOTTS, 30 ROCKEFELLER PLAZA, NEW YORK, NY, 10112, US

(21) Appl. No.: 10/492,384

(22) PCT Filed: Oct. 18, 2002
(86) PCT No.: PCT/US02/33387

Related U.S. Application Data
(60) Provisional application No. 60/335,210, filed on Oct. 19, 2001.

(51) Int. Cl. A61B 5/05

(52) U.S. Cl. 600/410

(57) ABSTRACT

Techniques for reducing the scan time required for the acquisition of two or more magnetic resonance imaging images of a subject having differing contrasts. In one arrangement, a combo acquisition protocol of N sets of parameters is selected prior to imaging. In order to image a subject, a first set of parameter values is selected from the protocol, a first RF pulse and at least one gradient field are used to excite the subject, a refocusing RF pulse and at least one gradient field is applied to the subject, a phase encoding gradient field is applied to the subject, and then a measurement gradient field is applied to the subject simultaneously while an induced signal is measured. The process is repeated to obtain N measurements, which are then processed into two or more reconstructed images of differing contrast, and where some of the measurements are used during the reconstruction of two or more of the images.

### CROSS-REFERENCE TO RELATED APPLICATIONS

This application is based on U.S. provisional patent application Ser. No. 60/335,210 filed Oct. 19, 2001, which is incorporated herein by reference for all purposes and from which priority is claimed.

### NOTICE OF GOVERNMENT RIGHTS

The U.S. Government has certain rights in this invention pursuant to the terms of the National Institute of Health award number R21CA85594.

### BACKGROUND OF THE INVENTION

1. Technical Field

The present invention relates generally to magnetic resonance imaging, and more particularly to techniques for reducing the scan time required to acquire multiple magnetic resonance images of different contrasts.

2. Background Art

Magnetic resonance imaging (“MRI”) is a well established imaging technique used in medical settings to produce images of the inside of the human body which permit a physician to discriminate between healthy and diseased soft tissue. Referring to FIG. 1, a simplified block diagram of a typical prior art MRI device is illustrated. The MRI device includes a main magnet 10, which provides a magnetic field B₀ that generates a steady magnetic field realizing a polarization of the nuclei of the protons of the specimen or subject for which an image is desired. Within magnet 10 there is a cavity or space in which the specimen or human to be examined is placed.

The apparatus also includes a gradient system for producing spatial linear gradient fields. These gradient fields are generally established by a set of three orthogonal direct current coils 11, 12 and 13, which generate the three principal gradients Gz, Gy, and Gx. These coils are driven by gradient generator 14, which in turn is controlled by a controller 16, which communicates with the host computer 20. Typical gradients used in MRI image acquisition are well known.

Existing MRI systems also generally include a radio frequency (RF) coil 17 which transmits a radio frequency field to the specimen being analyzed and senses a free induction decay or spin echo signal, which is induced after termination of the radio frequency pulse that is used for excitation. RF pulse unit 18 generates the RF pulse utilized to excite the specimen via RF coil 17. The signal processor 19 receives the small microvoltage level signals, which are reconstructed by computer 20 to form an image. The image is digitized and stored in the memory section of computer 20 for later display on display unit 21.

While existing MRI techniques are able to provide physicians with important clinical information, they are often criticized by patients who must spend considerable time with a portion of their body in an enclosed space, while maintaining a still body position, as well has by those responsible for paying for lengthily, and hence costly, patient visits. Accordingly, the reduction of scan time is an important issue in terms of both reducing medical costs and increasing patient throughput and comfort.

Several techniques have been developed to reduce the scan time needed to obtain a single MRI image. Advances in scanner hardware over the last twenty years have aided in reducing scan time by allowing the development of fast acquisition schemes, such as echo planar imaging (“EPI”) fast spin echo (“fast spin echo”) and fast gradient echo sequences. Most of these methods attempt to increase the amount of data acquired within one sequence or repetition time cycle, i.e. a larger portion of k-space is sampled before the next sequence cycle.

Other approaches to scan time reduction have focused on reducing the amount of k-space information that needs to be acquired to obtain an image, traversing k-space along different trajectories instead of a Cartesian grid, and utilizing variable acquisition parameters. For example, H. K. Song et al., “Variable TE gradient and spin echo sequences for in vivo MR microscopy of short T2 species,” Magnetic Resonance in Medicine, vol. 39, pp. 251-8 (1998) discloses a technique utilizing variable echo time to shorten the echo time for in vivo MR microscopy. Similarly, B. Kuhn et al., “Fast proton spectroscopic imaging employing k-space weighting achieved by variable repetition times,” Magnetic Resonance in Medicine, vol. 35, pp. 457-64 (1996) discloses the variation of repetition time to shorten total acquisition time in spectroscopic imaging.

Despite such improvements in acquisition speed, only one image of a particular contrast is generally acquired with each technique. If multiple images of differing contrasts are needed (e.g. a T₁- and a T₂-weighted image for clinical diagnosis), image acquisition must be repeated with different acquisition parameters. The resulting total scan time for all images is then the sum of the scan time for each image with a specific contrast. Moreover, often a new scan set-up, such as slice prescription, is required to obtain an additional image of a different contrast of the same geometry.

There have been several attempts to provide a technique for reducing the scan time required to acquire multiple magnetic resonance imaging images having differing contrasts. For example, K. Oshio et al, “Simultaneous acquisition of proton density, T1, and T2 images with triple contrast RARE sequence,” J. Computer Assisted Tomography, vol. 17, pp. 333-8 (1993) discloses the combination of two fast spin echo sequences (8-echo and 4-echo) with two different repetition times to obtain images of three different contrasts (T₁-, PD-, and T₂-weighted) in one acquisition. Likewise, B. A. Johnson et al., “Evaluation of shared-view acquisition using repeated echoes (SHARE): a dual-echo fast spin-echo MR technique,” Ajnr: American Journal of Neuroradiology, vol. 15, pp. 667-73 (1994) discloses the use of a dual-contrast fast spin echo sequence.

Unfortunately, such previous attempts fail to satisfactorily integrate the use variable acquisition parameters, k-space data sharing, and multi-contrast imaging, in order to reduce scan time for general clinical applications, and often require specific hardware as part of their implementations. Accordingly, there remains a need for a technique for reducing the scan time required to acquire multiple magnetic resonance imaging images having differing contrasts, which is able to overcome these shortcomings.

### SUMMARY OF THE INVENTION

An object of the present invention is to provide a technique for reducing the scan time required to acquire multiple magnetic resonance imaging images having differing contrasts.

Another object of the present invention is to provide techniques for utilizing one set of k-space data to generate two or more MRI images.

Yet another object of the present invention is to provider a MRI technique which integrates the concepts of variable acquisition parameters, k-space data sharing, and multi-contrast imaging, in order to reduce scan time for general clinical applications.

Still another object of the present invention is to provide a reduced scan time MRI technique, which is not dependent on a specific hardware configuration.

In order to meet these and other objects of the present invention which will become apparent with reference to further disclosure set forth below, the present invention provides techniques for reducing the scan time required to acquire two or more magnetic resonance imaging images of a subject having differing contrasts. In one arrangement, a combo acquisition protocol of N sets of parameters is selected prior to imaging. Each set of acquisition parameters is used to obtain a single measurement of the subject, and certain measurements are used to obtain more than one MR image. For example, when N is 384 and two images are obtained, 128 measurements are shared.

In order to image a subject, a first set of parameter values is selected from the protocol, which may control the amplitude, frequency and timing of all RF signals and gradient fields applied to the subject. Next, a first RF pulse and at least one gradient field are used to excite the subject, a refocusing RF pulse and at least one gradient field are applied to the subject, a phase encoding gradient field is applied to the subject, and then a measurement gradient field is applied to the subject simultaneously while an induced signal is measured. The process is repeated to obtain N measurements, which are then processed into two or more reconstructed images of differing contrast, where some of the measurements are used during the reconstruction of two or more of the images.

In a preferred process, the combo acquisition protocol is developed by selecting the number of desired image contrasts and the desired scan time reduction, and then designing the combo acquisition protocol by selecting a phase encoding scheme and parameter variation schemes, and then refining the phase encoding scheme based on the selected variation scheme.

In an especially preferred methodology, an index and multiple phase encoding gradient fields are employed to obtain a set of measurements. Advantageously, after a first RF pulse and at least one gradient field are used to excite the subject, an echo train index is set at an initial value, a refocusing RF pulse and at least one gradient field are applied to the subject, an index-specific phase encoding gradient field is applied to the subject, and then a measurement gradient field is applied to the subject simultaneously while an induced signal is measured. Next, the inverse of the phase encoding gradient field is applied, and a determination is made as to whether the index has been completed. The method is repeated until the all values of the index have been used for a particular set of protocol parameters, and then is repeated with different sets of protocol parameters.

The accompanying drawings, which are incorporated and constitute part of this disclosure, illustrate preferred embodiments of the invention and serve to explain the principles of the invention.

### BRIEF DESCRIPTION OF THE DRAWINGS

FIG. 1 is a simplified block diagram of a prior art MRI apparatus;

FIG. 2 is a flow diagram of a method in accordance with the present invention which may be performed on the apparatus of FIG. 1;

FIG. 3 is a flow diagram of a preferred method for selecting a combo acquisition protocol useful in the method of FIG. 2;

FIG. 4 is a flow diagram of a preferred method for obtaining k-space data utilized in the method of FIG. 2;

FIG. 5 is an illustrative diagram showing an exemplary phase encoding scheme for a 2-contrast combo acquisition with 384 sequence cycles;

FIGS. 6(a)-(b) are illustrative diagrams showing exemplary parameter variation schemes useful with the scheme of FIG. 5;

FIGS. 7 (a)-(b) are illustrative diagrams showing signal levels (k-space data weighting) obtained by the scheme of FIGS. 6(a)-(b);

FIGS. 8 (a)-(d) are illustrative diagrams showing exemplary images, FIGS. 8(a) and (c) being obtained by the schemes FIGS. 6 (a) and (b), and FIGS. 8 (b) and (d) from a corresponding standard acquisition technique;

FIGS. 9 (a)-(b) are illustrative diagrams showing exemplary parameter variation schemes useful for a 3-contrast spin echo combo acquisition with 608 sequence cycles;

FIGS. 10 (a)-(f) are illustrative diagrams showing exemplary images, FIGS. 10 (a), (c), and (e) being obtained by the schemes FIGS. 9 (a) and (b), and FIGS. 10 (b), (d), and (f) from a corresponding standard acquisition technique;

FIGS. 11 (a)-(c) are illustrative diagrams showing signal levels (k-space data weighting) obtained by the scheme of FIGS. 9 (a)-(b);

FIGS. 12 (a)-(b) are illustrative diagrams showing preferred parameter variation schemes useful for a 3-contrast fast spin echo combo acquisition with 160 sequence cycles;

FIGS. 13 (a)-(f) are illustrative diagrams showing exemplary images, FIGS. 13 (a), (c), and (e) being obtained by the schemes FIGS. 12 (a) and (b), and FIGS. 13 (b), (d), and (f) from corresponding standard acquisition techniques;

FIGS. 14 (a)-(c) are illustrative diagrams showing signal levels (k-space data weighting) obtained by the scheme of FIGS. 12 (a)-(b);

FIGS. 15 (a)-(f) are illustrative diagrams showing exemplary multiple slice images, obtained by the schemes FIGS. 12 (a) and (b);

FIG. 16 is a graph plotting residue quantities against acquisition protocols.

Throughout the FIGS., the same reference numerals and characters, unless otherwise stated, are used to denote like features, elements, components or portions of the illustrated embodiments. Moreover, while the present invention will now be described in detail with reference to the FIGS., it is done so in connection with the illustrative embodiments.

### DETAILED DESCRIPTION OF THE PREFERRED EMBODIMENTS

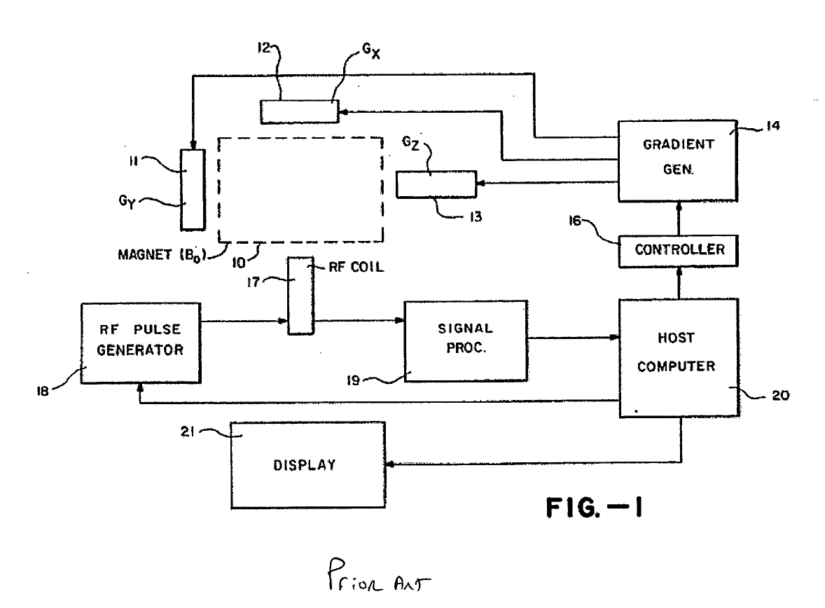

The techniques of the present invention may be used with standard MRI apparatus if parameters of the radio frequency (“RF”) pulse train and gradient may be controlled by, and feedback provided to, a host computer or other controlling mechanisms. For example, the system described above with reference to FIG. 1 provides such a suitable apparatus.

The techniques described below may be incorporated into such an MRI apparatus by way of programming the host computer 20 and/or controller 16 in order to select a control protocol, control the generation of desired waveforms from the RF pulse generator 18 and gradient generator 14, and measure signals that are induced in the subject being imaged. The software is generally written in any number of conventional programming languages and can be stored and transported on conventional computer readable media, such as magnetic storage disks (floppy diskettes, hard disks and the like), optical disks (CD-ROMS) and the like.

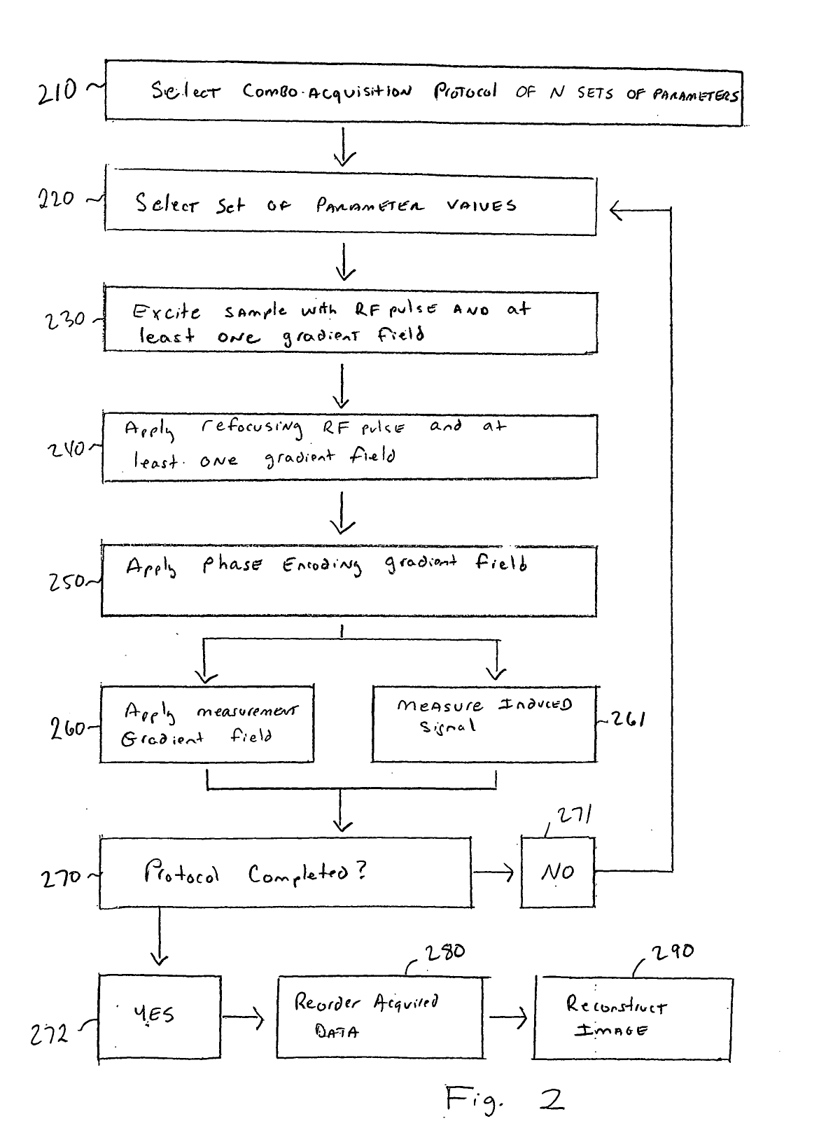

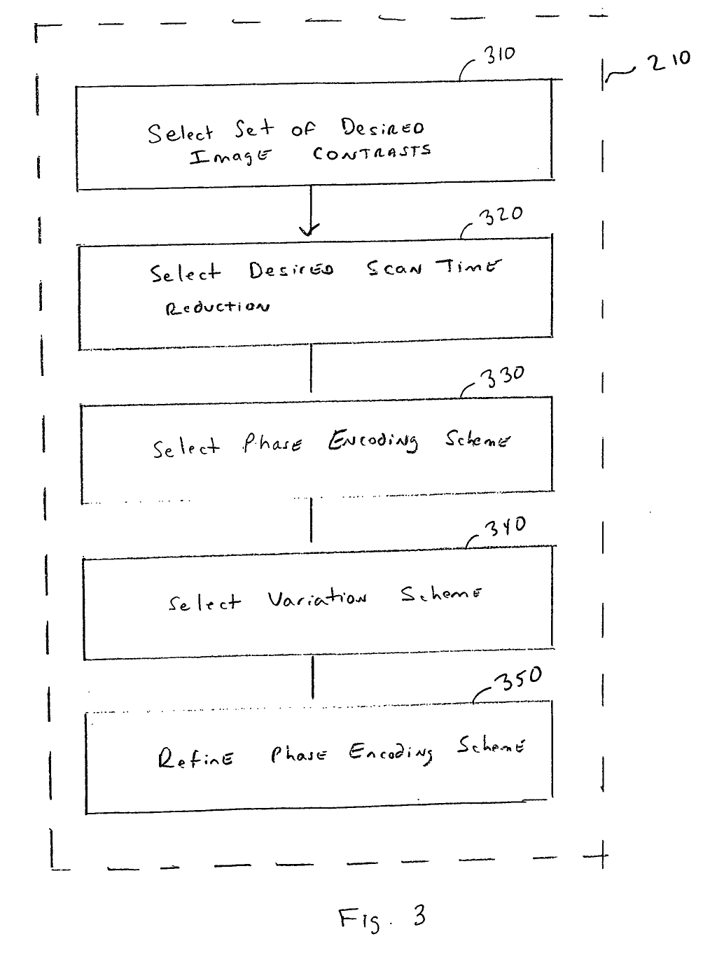

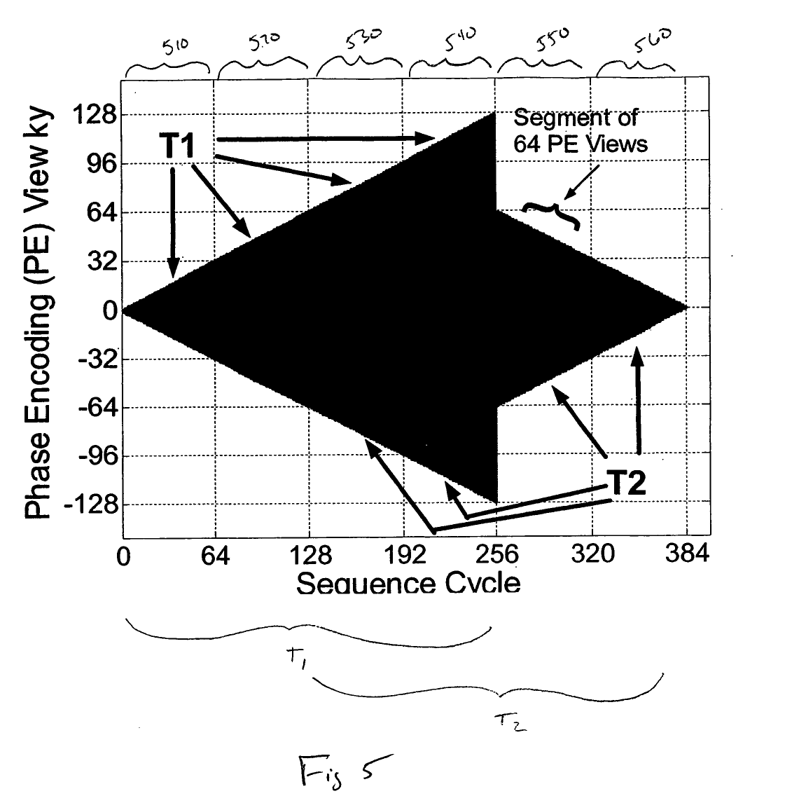

Referring to FIG. 2, an exemplary method in accordance with the present invention will be explained. First, a combo acquisition protocol of N sets of parameters is preferably selected 210. The details of this selection process, which may manual or automated, are described below, e.g., with reference to FIG. 3. In accordance with the present invention, the protocol contains N sets of acquisition parameters suitable for obtaining two or more MRI images of the subject having differing contrast. Each set of acquisition parameters is used to obtain a single measurement of the subject, and certain measurements are used obtain more than one of MRI images. For example, in one preferred embodiment described below with reference to FIG. 5, N is 384, and 128 measurements are shared in obtain two different contrast images of a subject.

In step 220, a first set of parameter values is selected from the protocol. As described in more detail below, the parameters control the amplitude, frequency and timing of all RF signals and gradient fields applied to the subject during steps 230-270, as well as timing of the measurement of the induced signal.

With the acquisition parameters set, a first RF pulse and at least one gradient field are used to excite the subject 230. As those skilled in the art will appreciate, while use of an RF pulse permits the primary imaging of the subject, the gradient fields are used to spatially encode an image. In the next steps, a refocusing RF pulse and at least one gradient field is applied to the subject 240, a phase encoding gradient field is likewise applied to the subject 250, and then a measurement gradient field is applied to the subject 260 simultaneously while the induced signal is measured through the RF coil 261.

In step 270, a determination is made as to whether the protocol has been completed, i.e., if N measurements with appropriate acquisition parameters have been obtained. If another measurement is required 271, a new set of acquisition parameter values is selected 220, and the process 220-270 repeated. If all N measurements have been completed 272, the process may continue to post-measurement processing such as the reordering of the acquired data 280, and the display of the reconstructed images 290, e.g., on a display 21. With respect to the reordering of data 280, the exact ordering of all phase encoding views within the segments of a phase encoding scheme may be dependent on the particular set of parameter variation curves of a combo acquisition

Referring to FIG. 3, a preferred method for the step of selecting a combo acquisition protocol 210 is described. A set of desired image contrasts is selected 310, either by a user or, in an automated process, by the host computer 20 depending on the MRI imaging to be conducted and/or the subject to be scanned. In the preferred embodiments discussed below, the number of image contrasts is set at two or three, but the principals of the present invention are readily extended to obtain four or more image contrasts.

Next, the desired scan time reduction is selected, either by a user or, in an automated process, by the host computer 20 depending on the MRI imaging to be conducted and/or the subject to be scanned. In the preferred embodiments discussed herein, when the number of image contrasts is set at two, the scan time reduction is set at 50%, and when the number of image contrasts is set at three, the scan time reduction may be set at 30%. However, the principals of the present invention fully apply to other scan time reductions.

With the desired image contrasts and scan time reduction selected, the combo acquisition protocol may be designed through the selection of a phase encoding scheme 330 and variation scheme 340, and the refinement of the phase encoding scheme 350 based on the selected variation scheme. Steps 330-350 are described in further detail below for preferred processes where the number of image contrasts is two and scan time reduction is set at 50%, and when the number of image contrasts is three and the scan time reduction is set at 30%.

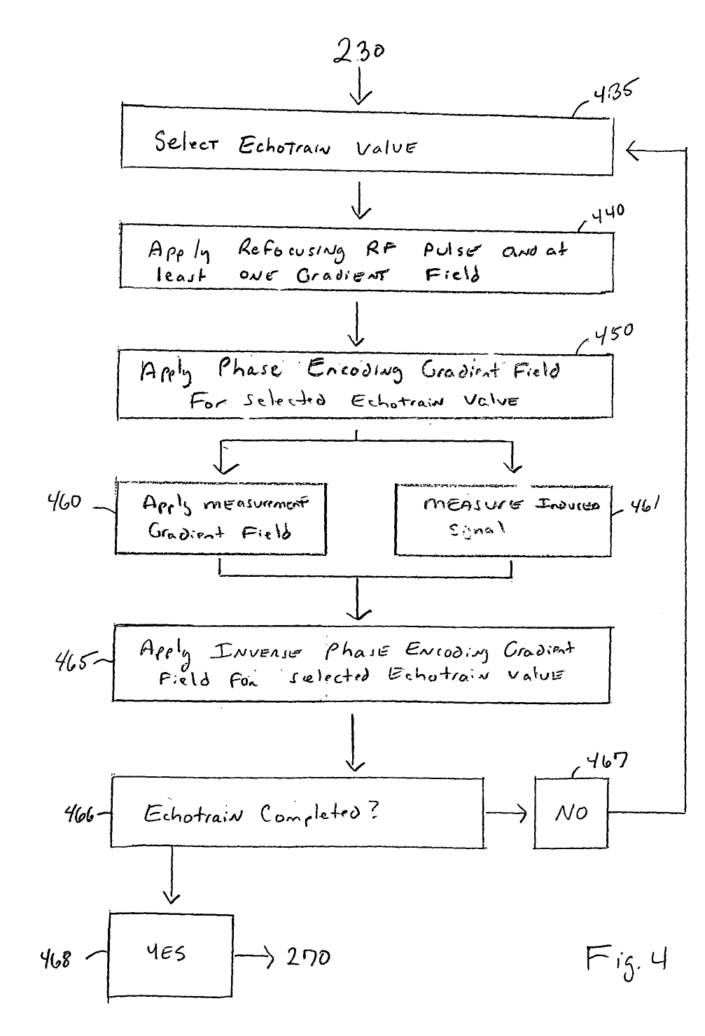

Referring next to FIG. 4, a particularly advantageous method for obtaining k-space data will be described. This method is similar to that described with reference to FIG. 2, except that it makes use of an index and multiple phase encoding gradient fields to obtain multiple measurements, and thus may be used to further reduce scan time.

As in FIG. 2, after a first RF pulse and at least one gradient field are used to excite the subject 230, the echo train index is set at an initial value 435, a refocusing RF pulse and at least one gradient field is applied to the subject 440, a phase encoding gradient field is likewise applied to the subject 450, and then a measurement gradient field is applied to the subject 460 simultaneously while the induced signal is measured through the RF coil 461. However, the phase encoding gradient field is dependent on the particular echo train index value. Next, the inverse of the phase encoding gradient field is applied 465, to remove the influence of that field. In step 466, a determination is made as to whether the index has been completed, and if so, the process continues to step 270. If the index is not completed, 467, the next index value is selected 435, and steps 440-466 are repeated.

The steps 330, 340, 350 involved in selecting a combo acquisition protocol 210 shall next be described in more detail. As noted above, a combo acquisition protocol is preferably formed 210 through the selection of appropriate phase encoding and variation schemes, the refinement or optimization of the phase encoding scheme, and in some cases, the reordering of acquired data 280.

A phase encoding scheme permits a selection of the amount and distribution (spatial frequency content) of k-space data sharing between images of different contrasts in combo acquisitions. An example of such a scheme for a 2-contrast spin echo combo acquisition is shown in FIG. 5, which includes six segments of 64 phase encoding views. The first four segments 510, 520, 530, 540 may be used to reconstruct a T₁-weighted image, and the last four segments 530, 540, 550, 560 may be used to reconstruct a T₂-weighted image having a different contrast. Those skilled in the art should appreciate that a spin echo combo acquisition may be subdivided in other ways consistent with the teachings of the present invention, e.g., the acquisition shown in FIG. 5 could be divided into segments of 32, 16, 8 or other number of phase encoding views.

Stepping through all phase encoding numbers may be carried out in zigzag fashion, i.e. N_{phase encoding}= . . . 0, −1, 1, −2, 2, . . . , (+N_{y}/2−1), (−N_{y}/2), which is a particular example for the exact ordering of all phase encoding views within the segments of a phase encoding scheme (refinement of phase encoding scheme).

To obtain different contrast weightings in combo acquisitions and to reduce scan time of combo acquisitions, appropriate variation schemes should be selected in accordance with the selection of variable acquisition parameters. For example, the acquisition parameters of repetition time and echo time may be varied for spin echo combo acquisitions, and the parameters of repetition time, echo spacing (“ESP”), and echo train length (“ETL”) may be varied in fast spin echo combo acquisitions. It should be noted that the variation of echo spacing parameter essentially corresponds to the use of variable echo time in spin echo imaging. More precisely, echo spacing is the fixed time interval between different echoes and it defines the timing for all echoes in a echo train by TEₚ=p·ESP, where p is the number of an echo and p=1, 2, . . . , ETL . The effective echo time TE_{eff} for a fast spin echo sequence is then determined by the echo time, at which the zero phase encoding view (k_{y}=0) for an image is acquired.

The range of parameter variation depends on the choice of different contrasts to be obtained in a multi-contrast combo scan. For example, to obtain images of all three contrasts in a spin echo combo acquisition, repetition time may be varied from 500 ms to 2500 ms, and echo time from 20 ms to 150 ms. However, the variation of acquisition parameters does not necessarily have to be continuous to preserve contrast and to yield images with few artifacts.

Moreover, since combo acquisitions are intended for scan time reduction in a clinical setting, parameter variation schemes should be designed to accommodate multi-slice imaging. Thus, for a 3-contrast combo acquisition, the minimum number of slices that can be acquired in each sequence cycle can be chosen to be N_{slices}=15. The constraint of multi-slice imaging then limits the range for parameter echo time for a given repetition time in spin echo combo scans and for parameter echo train length for given repetition time and echo spacing in fast spin echo combo acquisitions. For instance, with the requirement of 15 slices for a fast spin echo combo acquisition, and with the choice of echo spacing =15 ms and repetition time =500 ms, echo train length cannot exceed 2.

After appropriate phase encoding and variation schemes have been selected, it may be advantageous to refine the phase encoding and variation schemes to preserve desired contrast and minimize visible artifacts in the resulting images. In combo acquisitions, artifacts in the image domain are inevitably introduced through non-uniform data weighting in k-space, which, in turn, are caused by varying selected acquisition parameters over all phase encoding views of a particular image. Accordingly, a preferred technique for optimizing a combo acquisition protocol involves the preservation of contrast, the analysis of signal levels (data weighting in k-space) and the minimization of three quantitative criteria: the energy of the “residue point spread function” E (PSFᵣₑₛ), the energy of “residue profiles” across sharp tissue boundaries E(profilesᵣₑₛ), and the energy of “residue images” E(imagesᵣₑₛ).

Regarding the preservation of image contrast, the zero phase encoding view (k_{y}=0) for each image in a combined acquisition may be acquired with specific settings for selected acquisition parameters, such as repetition time and echo time. These settings should be the same as those used to obtain a specific contrast in a corresponding standard acquisition. In general, low frequency phase encoding views with |k_{y}|<(|k_{y,max}|/2) are acquired with values close to these settings for contrast preservation. To shift from one contrast weighting to another, acquisition parameters are rapidly varied during the acquisition of high frequency phase encoding views with |k_{y}|>(|k_{y,max}|/2). These phase encoding views contribute less to the contrast, but carry significant and subtle details of an image.

Regarding the analysis of signal levels, signal levels (magnitudes) for a single object point of a particular tissue should be generated, with data being separately acquired with phase encoding set to zero using selected parameter variation schemes. Signal levels correspond to data weighting in k-space with respect to phase encoding views k_{y}|ₖ_{ₓ}₌₀. The set of signal levels of all tissues for each image according to a selected parameter variation scheme is then analyzed, and variation curves for acquisition parameters modified, where possible, to preserve the order of signal levels for a particular contrast, e.g. |signal_{WM}|>|signal_{GM}|>|signal_{CSF}| for a T₁-weighted image, and to minimize discontinuities between signal levels for different segments of phase encoding views.

The point spread function (“PSF”) for MR can be considered as the inverse Fourier transform of a filter H(\vec{k}) that multiplies the k-space data. In the image domain this is equivalent to convolution of the transformed data with a filter h(\vec{r}), where h is the point spread function . The energy of the “residue PSF” E(PSFᵣₑₛ), i.e. the energy of the difference in PSF between combo and corresponding standard acquisitions, is defined as (for phase encoding along the y-axis):

$$E ( {PSF}_{res} ) = {\sum }_{i = 1}^{Ncontrast} {\sum }_{j = 1}^{{N}_{tissues}} {w}_{ij}^{PSF} \cdot {\int }_{- {FOV}_{y} / 2}^{+ {FOV}_{y} / 2} {\vert {PSF ( y )}_{ij}^{combo} - {PSF ( y )}_{ij}^{standard} \vert}^{2} ⅆ y \qquad ( 1 )$$

In Eq. 1, N_{contrast} is the number of different contrasts in a multi-contrast acquisition, Nₜᵢₛₛᵤₑₛ is the number of tissues used in phantom data, and wᵢⱼ^{PSF} are the weighting factors that depend on the respective diagnostic importance of a contrast, and on the signal level from the corresponding standard acquisition of a tissue for each contrast. These weighting factors account for the “visibility” of artifacts for different tissues in images of a particular contrast. For example, artifacts in tissues with larger signal levels for a specific contrast are usually more visible and thus have to be weighted more strongly.

Changes of the point spread function do not always directly translate into changes of the visual appearance of objects in an image, e.g. for homogeneous tissue areas. However, the second quantitative criterion, the energy of residue profiles, is derived from image profiles across sharp tissue boundaries (edges). Such profiles can be generated, e.g., from images of a phantom of two adjacent rows of three consecutive homogeneous tissue blocks of size 60×30 units along in-plane coordinates. To yield a quantitative criterion, the energy of “residue profiles” across sharp tissue boundaries E (profilesᵣₑₛ) may be expressed as

$$E ( {profiles}_{res} ) = {\sum }_{i = 1}^{Ncontrast} {\sum }_{j = 1}^{{N}_{tissueComb}} {w}_{ij}^{profiles} \cdot {\int }_{- {FOV}_{y} / 2}^{+ {FOV}_{y} / 2} {\vert {profile ( y )}_{ij}^{combo} - {profile ( y )}_{ij}^{standard} \vert}^{2} ⅆ y \qquad ( 2 )$$

where, N_{tissueComb} refers to the number of tissue combinations. All other notations are as defined for E(PSFᵣₑₛ) in (1).

The third quantitative criterion for assessing the optimization of combo acquisitions, the energy of “residue images” E(imagesᵣₑₛ), is the weighted sum of the energies of the difference images obtained by subtracting the images from combo and corresponding standard acquisitions from each other:

$$\begin{matrix}E ( {images}_{res} ) = {\sum }_{i = 1}^{Ncontrast} {w}_{i}^{images} \cdot \\ { {\int \int }_{{FOV}_{i n \text{-} plane}} \vert {image ( x , y )}_{i}^{combo} - {image ( x , y )}_{i}^{standard} \vert}^{2} ⅆ x ⅆ y\end{matrix} \qquad ( 3 )$$

where the summation of energies is carried out over all images of different contrasts weighted by factors wᵢ^{images}. Optimization of combo acquisition is achieved by minimizing all three quantitative criteria, since minimization of these criteria corresponds to a reduction of artifacts. Modifications of phase encoding and variation schemes to optimize combo acquisitions should therefore be kept or rejected based on their effect on these criteria.

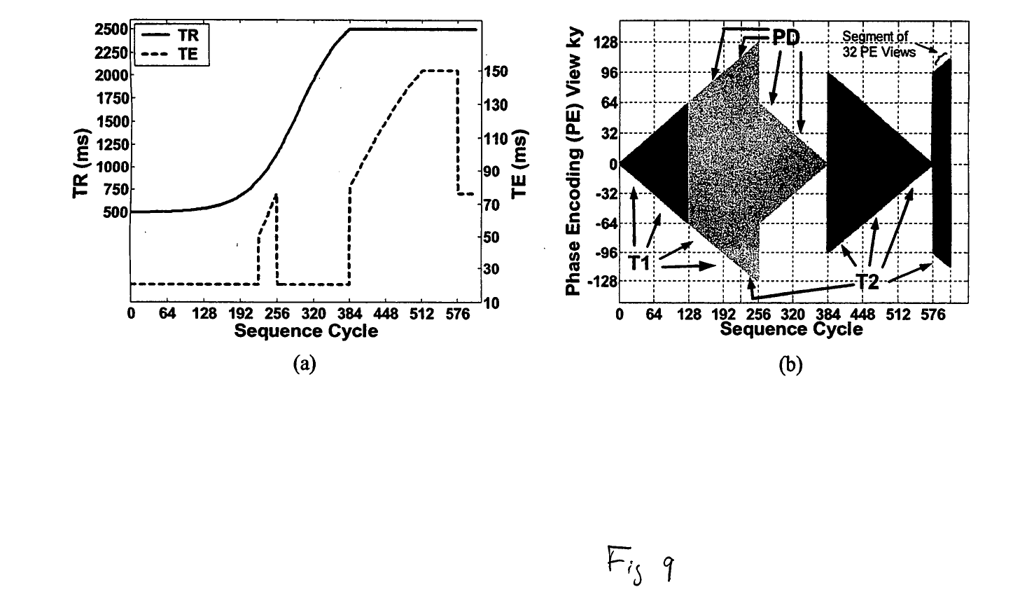

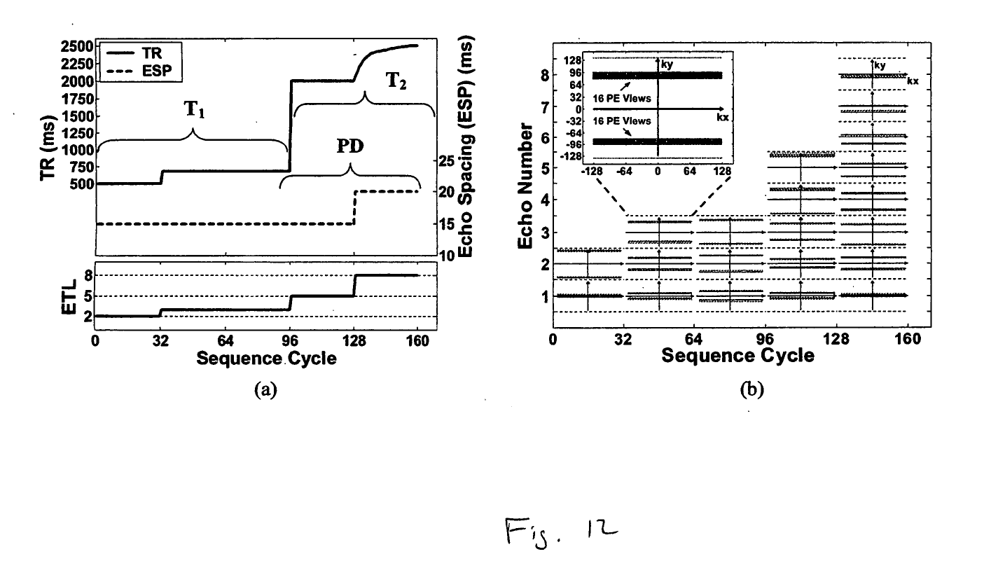

In FIGS. 5-14, three exemplary preferred embodiments of the present invention will be described, with FIGS. 5-8 describing a two contrast spin echo combo acquisition protocol, FIGS. 9-11 describing a three contrast spin echo combo acquisition protocol, and FIGS. 12-14 describing a three contrast fast spin echo combo acquisition protocol. For each embodiment, reconstructed MRI images are compared to images obtained by corresponding standard acquisition techniques. The latter served as references in terms of image contrast, quality, and resolution. Images shown are simulated magnitude images.

For the standard acquisitions for spin echo combo acquisitions, images of the three different contrasts (T₁-, (PD)-, and T₂-weighted) were acquired using a single repetition time and a single echo time. The choices of acquisition parameters repetition time and echo time for these contrasts are summarized in Table I (NEXis assumed to be 1). For 3-contrast fast spin echo combo acquisitions, a combination of a spin echo sequence for the T₁-weighted image and a dual-contrast fast spin echo sequence with no data sharing for the other two images was selected as standard acquisitions. Parameters for these two scans are also included in Table I. Note that the scan time reduction of combo acquisitions depends on the choice of the corresponding standard acquisitions used as a reference.

TABLE I

| SE Standard Acquisxitions | | | FSE Standard Acquisitions | | | |
|---|---|---|---|---|---|---|
| Contrast | TR (ms) | TE (ms) | ETL | ESP (ms) | TR (ms) | TE_{eff} (ms) |
| T₁ | 500 | 20 | 1 | 15 | 500 | 15 |
| PD | 2500 | 20 | 8 | 20 | 2500 | 20 |
| T₂ | 2500 | 150 | 8 | 20 | 2500 | 140 |

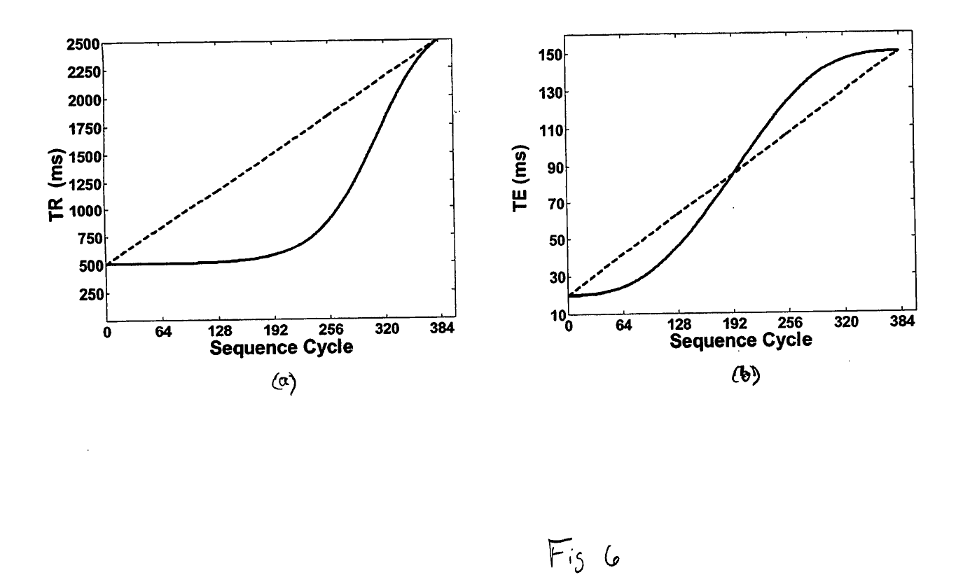

Referring next to FIGS. 6-8, a first embodiment of the invention will be FIGS. 6 (a)-(b) show an exemplary parameter variation scheme useful with the described. phase encoding scheme described above with reference to FIG. 5. In order to optimize the combo acquisition protocol, several sigmoidal and sinusoidal functions were used to vary repetition time and echo time, with sigmoidal variation curves expressed as:
f_{sigm}(s)=c₁+c₂·sigm(s) sigm(s)=(1+e^{-s})⁻¹ (4)
with s ε[a,b].

Parameters c₁ and c₂ are constants depending on the minimum and maximum values of the desired variation. Changing these constants and the interval [a,b] yields different sigmoidal functions. One choice for a variation scheme that yields results without major artifacts is shown in FIGS. 6 (a)-(b), with parameters for the curve for repetition time in FIG. 6(a) chosen as follows:

$$\begin{matrix}{c}_{1} = {TR}_{max} - \frac{( {TR}_{max} - {TR}_{min} ) \cdot sigm ( b )}{( sigm ( b ) - sigm ( a ) )} & ( 5 ) \\ {c}_{2} = \frac{( {TR}_{max} - {TR}_{min} )}{( sigm ( b ) - sigm ( a ) )} & \\ a = - 9 , b = ln ( 9 ) . & \end{matrix}$$

For the sinusoidal curve for echo time in FIG. 6(b), the analytical formulation is

$${f}_{sin} ( s ) = {TE}_{min} + \frac{{TE}_{max} - {TE}_{min}}{( {N}_{cycles} - 1 )} \cdot s - 20 \cdot sin ( s \cdot \frac{2 π}{( {N}_{cycles} - 1 )} ) , \qquad ( 6 )$$

where s ε[0, (N_{cylces}−1)] and N_{cycles}=384 is the total number of sequence cycles.

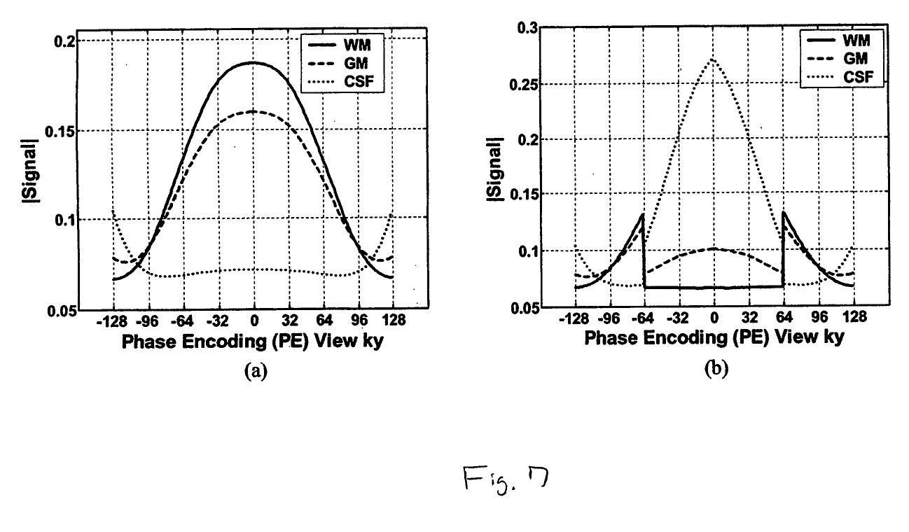

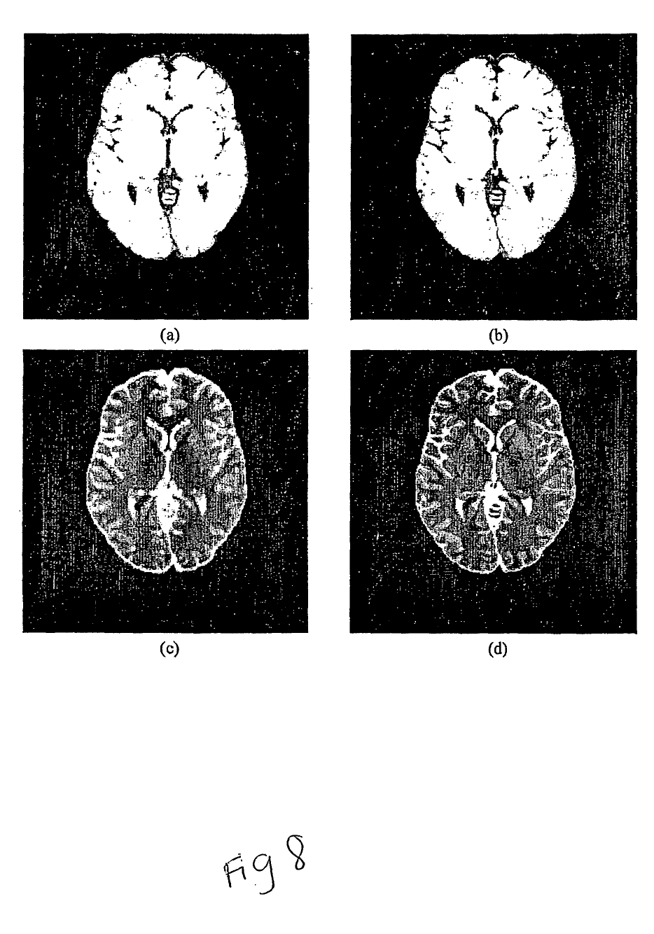

FIGS. 7 (a)-(b) show signal levels obtained by the scheme of FIGS. 6 (a)-(b). FIGS. 8 (a)-(d) show exemplary images, with FIGS. 8(a) and (c) being obtained by the schemes FIGS. 6 (a) and (b), and FIGS. 8 (b) and (d) from a corresponding standard acquisition technique. Comparing the images in FIGS. 8(a) and (c) with the corresponding images from standard acquisitions shown in FIGS. 8(b) and (d), respectively, illustrates the preservation in contrast. The T₁-weighted image in FIG. 8(a) shows only minor brightening of cerebrospinal fluid (CSF) and nearly no ringing artifacts, though the T₂-weighted image in FIG. 8 (c) shows some minor ringing within the areas of white matter (WM) and gray matter (GM). Fine structures of tissues WM and GM though were blurred in the T₁-weighted image due to relatively small signal levels for phase encoding views with |k_{y}|>96 as shown in FIG. 7(a) that resulted in a widened PSF. A similar observation holds for CSF features in the T₂-weighted image.

For this specific 2-contrast spin echo combo acquisition, a scan time reduction of 52% was achieved relative to the scan time needed for two separate standard acquisitions. A similar scheme using sigmoidal functions to vary both repetition time and echo time produces a 47% scan time reduction, a T₁-weighted image that is slightly more blurred, and an improved T₂-weighted image exhibiting less ringing artifacts. This illustrates a trade-off between the optimization of one contrast weighting at the cost of another.

Referring next to FIGS. 9-11, a three contrast spin echo combo acquisition protocol is described. FIGS. 9 (a)-(b) show an exemplary parameter variation scheme useful for a 3-contrast spin echo combo acquisition with 608 sequence cycles. A set of specific variation curves for repetition time and echo time resulting from these experiments is shown in FIG. 9(a). The corresponding phase encoding scheme is shown in FIG. 9(b), with arrows indicating portions of acquired data used to reconstruct three images of T₁-, PD- and T₂-contrast, respectively.

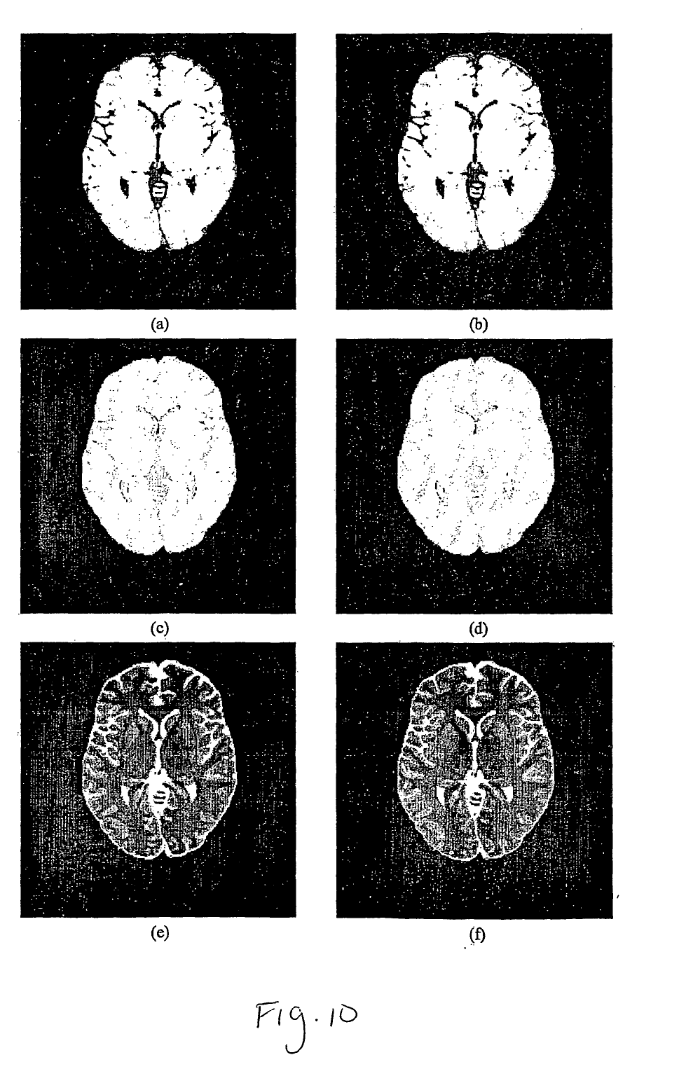

FIGS. 10 (a)-(f) show exemplary images, with FIGS. 10 (a), (c) and (e) being obtained by the schemes FIGS. 9 (a) and (b), and FIGS. 10 (b), (d) and (f) from a corresponding standard acquisition technique. As seen in FIG. 10, images from the spin echo combo acquisition have sufficient contrast definition and do not exhibit significant artifacts. For example, the T₁- and the T₂-weighted images in FIGS. 10 (a) and (e) are very similar to their counterparts from standard acquisitions shown in FIGS. 10 (b) and (f), respectively. The images were acquired with 31% less scan time compared to standard acquisitions.

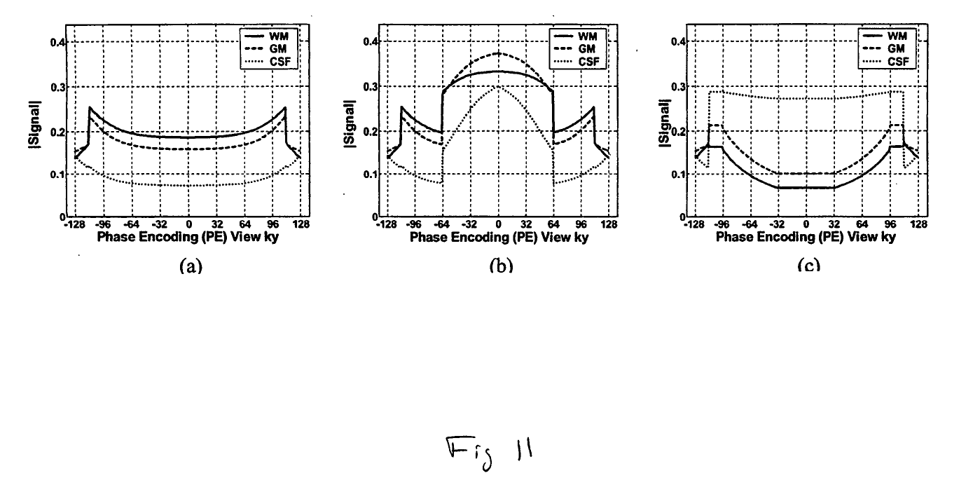

FIGS. 11 (a)-(c) show signal levels obtained by the scheme of FIGS. 9 (a)-(b). As can be seen from FIGS. 11 (a) and (c), the order of signal levels for the corresponding contrasts is preserved for almost all phase encoding numbers of the T₁- and the T₂-weighted images. Only signal levels for the highest frequencies are attenuated when compared to the corresponding standard acquisitions. This explains the similarities between the images from combo and standard acquisitions. For the PD-weighted image, signal levels of higher frequencies (|k_{y}|>64) were more strongly attenuated, and their order for the three tissues was modified from the one for PD-contrast as shown in FIG. 11 (b). Thus, the PD-weighted image exhibits a slightly different visual appearance (darker GM) than its counterpart from a standard scan in FIG. 11 (d).

Is should be noted that in order to correct for different data weighting in k-space, various filters derived from the distribution of signal levels over all phase encoding views according to a selected variation scheme may be applied. But naive filtering, which may realize an enhancement of the signal for certain tissue types, is usually not beneficial for other tissue types.

Referring next to FIGS. 12-14, a three contrast fast spin echo combo acquisition protocol is described. Combo acquisitions using the fast spin echo sequence and achieving scan time reduction make this technique more practical and more useful. As with spin echo combo scans, initial acquisition protocols for 3-contrast fast spin echo combo acquisitions should be refined and optimized based on the principles described above.

FIGS. 12 (a)-(b) show a preferred parameter variation scheme useful for a 3-contrast fast spin echo combo acquisition with 160 sequence cycles. Optimized variation schemes for parameters repetition time (top, solid), echo spacing (ESP) (top, dashed), and echo train length (ETL) (bottom, solid) are presented in FIG. 12(a). Data sharing between images is illustrated through parentheses that essentially comprise the particular set of MR signals used to reconstruct a T₁-, PD, or a T₂-weighted image, respectively.

As shown in FIG. 12 (a), data for the T₁-weighted image is mainly acquired with short repetition time and short echo time (i.e. from early echoes). Signals from mainly early echoes and longer repetition time are assigned to the PD-weighted image, whereas data from later echoes, i.e. long echo time, and long repetition time, are used for the reconstruction of the image with T₂-contrast. Note that parameter ESP was increased for the last sequence cycles of the fast spin echo combo acquisition to obtain increased T₂-contrast for the low spatial frequencies of the T₂-weighted image. The corresponding phase encoding scheme for this acquisition is shown in FIG. 12 (b) by marking the phase encoding views in k-space that are acquired with each echo for sets of 32 sequence cycles. An inset in the upper left corner of FIG. 12 (b) shows an enlarged k-space diagram for one echo.

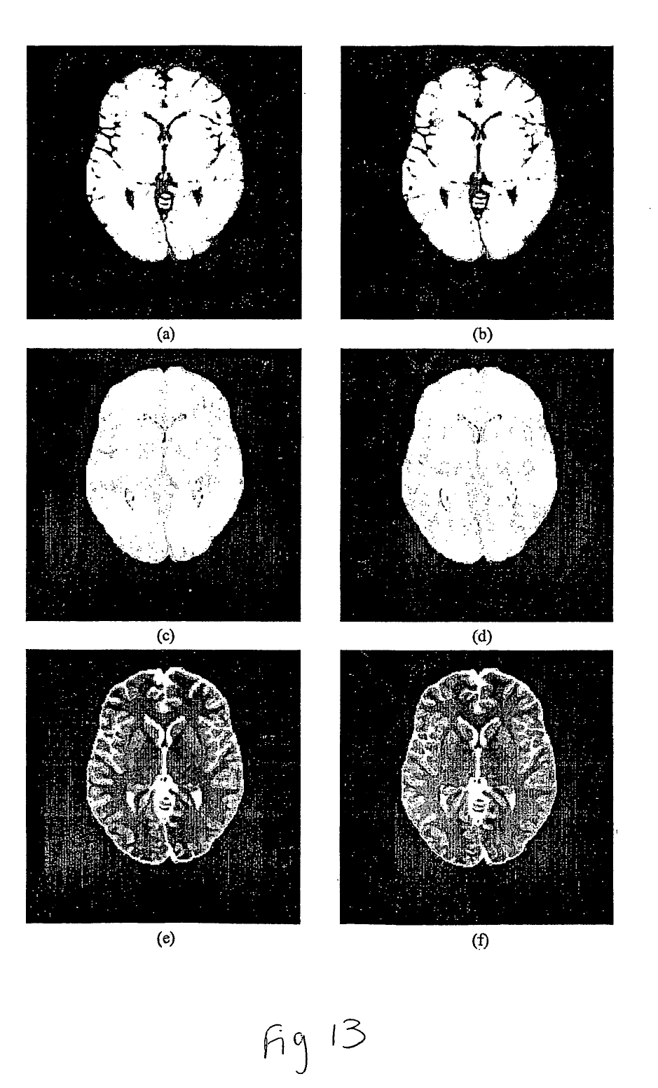

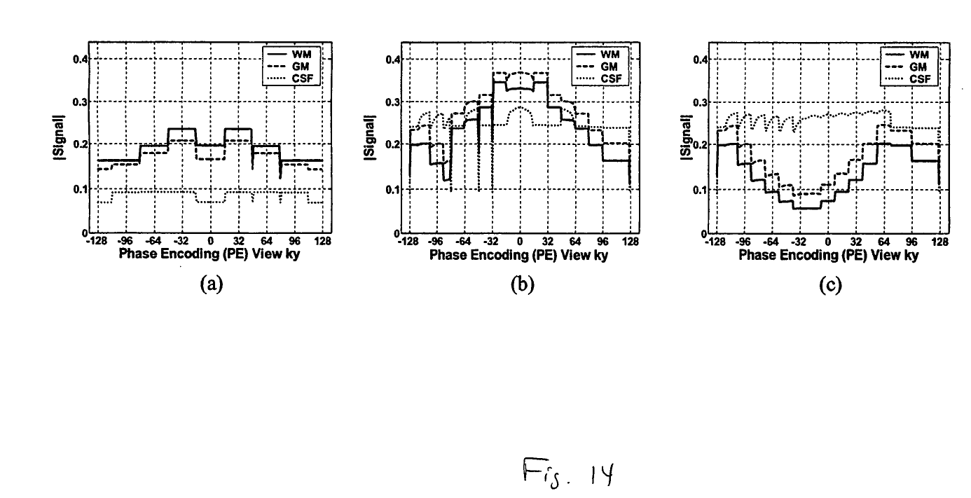

FIGS. 13 (a)-(f) show exemplary images, FIGS. 13 (a), (c) and (e) being obtained by the schemes FIGS. 12 (a) and (b), and FIGS. 13 (b), (d) and (f) from corresponding standard acquisition techniques. FIGS. 14 (a)-(c) show signal levels obtained by the scheme of FIGS. 12 (a)-(b). As shown in the FIGS., image contrast is well preserved, and artifacts have been significantly suppressed through semi-empirical optimization of the acquisition protocol. Similar to the results of the 3-contrast spin echo combo acquisition in FIG. 10, the T₁- and the T₂-weighted images in FIG. 13(a) and (e) closely resemble their counterparts from standard acquisitions in FIG. 13(b) and (f), respectively. Minor remaining artifacts in the T₁-weighted image in FIG. 13(a), such as faint ghosting in the WM caudal to the corpus callosun, are caused by non-uniform data weighting arising from data acquisition with variable ETL>1 and variable repetition time (see FIG. 14(a)).

The excellent image quality of the T₂-weighted image in FIG. 13(c) is the consequence of the data weighting shown in FIG. 14 (c), which is very similar to the data weighting for a conventional fast spin echo scan with ETL=8 and TE_{eff}=7.ESP. Sufficient T₂-contrast in the T₂-weighted image of the combo acquisition was attained by increasing ESP for the last 20 sequence cycles. Non-uniform and non-monotonic signal levels for the PD-weighted image shown in FIG. 14 (b), especially for tissues WM and GM for ky<(−80), resulted in some blurring and smearing of fine details. Similar artifacts though are also seen in the PD-weighted image in FIG. 13 (d).

The artifacts are largely caused by the non-uniform data weighting due to T₂-decay inherent to fast spin echo sequences with ETL>1. The additional modulation of signal levels induced by varying ETL and repetition time do not have a large impact in this case. For the images of a 3-contrast fast spin echo combo acquisition shown in FIG. 13 scan time is reduced by 30% when compared to a combination of a spin echo sequence for the T₁-weighted image and a dual-contrast fast spin echo sequence with no data sharing for the other two images. If one fourth of the acquired phase encoding views are shared between the two images of the dual-contrast fast spin echo sequence, scan time are reduced by 25%. Scan time reductions for the spin echo and fast spin echo combo acquisitions presented and for additional combinations not shown are summarized in Table II. It should be noted that the precision of the values in Table II refers to controlled simulation studies. In practice, these numbers might vary slightly with the number of phase encoding views actually used for MR data acquisition.

TABLE II

| | | 3-Contrast SE | 3-Contrast fast spin |
|---|---|---|---|
| Contrasts | 2-Contrast SE Combo | Combo | echo Combo |
| T₁-T₂ | 47%, 52% | — | — |
| T₁-PD | 47%, 52% | — | — |
| PD-T₂ | 25% | — | — |
| T₁-PD-T₂ | — | 31% | 30% |

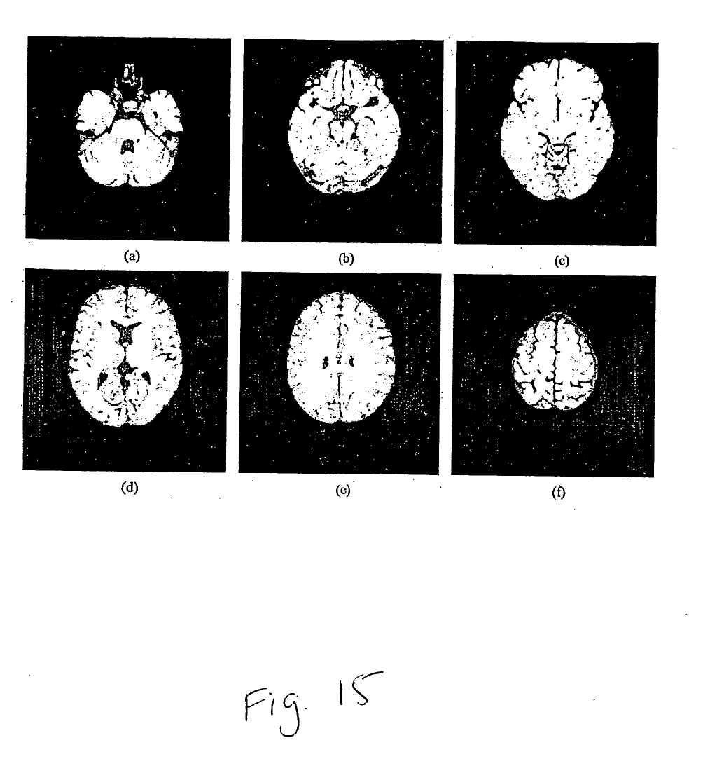

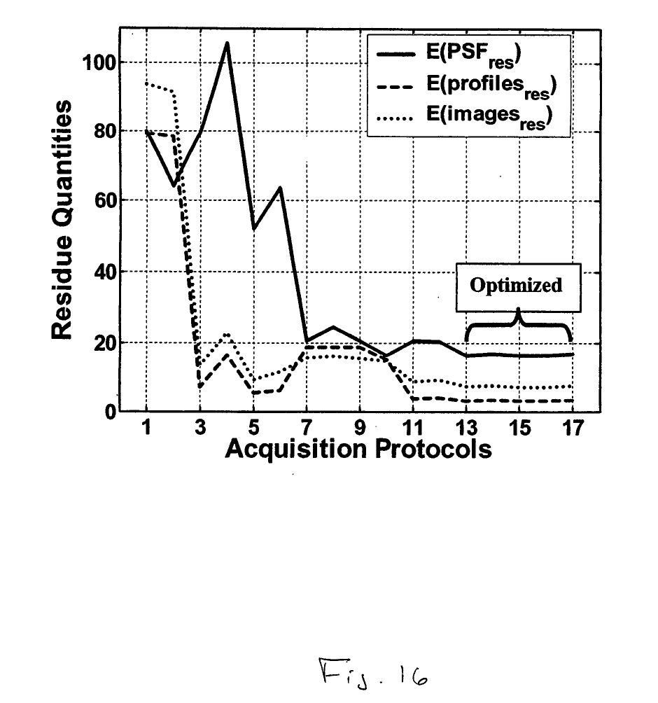

Referring next to FIGS. 15-16, several advantages of the present invention will now be described. T₁-weighted images of six axial slices of phantom data with tissues WM, GM, and CSF and acquired with the 3-contrast fast spin echo combo acquisition protocol described above with reference to FIG. 12, are presented in FIG. 15. Images in FIGS. 15(a)-(f) correspond to slice locations z=−30, −20, −10, +10, +30, +60, respectively image quality and level of artifacts are very similar to the ones in the image shown in FIG. 13(a) acquired with the same variation scheme and corresponding to slice location z=0. This shows that parameter variation and phase encoding schemes optimized for a specific set of phantom data yield similar results, when applied to phantom data with the same tissue types, but different (brain) structures. It should be noted though that the level of artifacts for these results depends on the spatial frequency content of the selected set of phantom data.

Values for the three quantitative criteria energy of residue PSF E (PSFᵣₑₛ), energy of residue profiles across sharp tissue boundaries E (profilesᵣₑₛ), and energy of residue images E(imagesᵣₑₛ) used to assess the optimization of combo acquisitions, are shown in Table III. The corresponding weighting factors used to compute these quantities are presented in Table IV.

TABLE III

| | | | 3-Contrast |
|---|---|---|---|
| | 2-Contrast SE | 3-Contrast SE | fast spin |
| Criterion | Combo | Combo | echo Combo |
| E (PSFᵣₑₛ) | 0.0312 | 0.0512 | 0.0073 |
| E (profilesᵣₑₛ) | 0.0244 | 0.0690 | 0.0330 |
| E (imagesᵣₑₛ) | 18.029 | 10.400 | 7.607 |

TABLE IV

| | | 2-Contrast SE | 3-Contrast SE | 3-Contrast fast |
|---|---|---|---|---|
| Criterion | Contrast | Combo | Combo | spin echo Combo |
| E (PSFᵣₑₛ) | T₁ | 1.0, 0.86, 0.38 | 1.0, 0.86, 0.38 | 1.0, 0.85, 0.36 |
| | PD | — | 0.89, 1.0, 0.80 | 0.91, 1.0, 0.78 |
| | T₂ | 0.24, 0.37, 1.0 | 0.24, 0.37, 1.0 | 0.27, 0.41, 1.0 |
| E (profilesᵣₑₛ) | T₁ | 1.0, 0.23, 0.30 | 1.0, 0.23, 0.30 | 1.0, 0.24, 0.32 |
| | PD | — | 0.85, 1.0, 0.46 | 1.0, 0.81, 0.45 |
| | T₂ | 1.0, 0.17, 0.21 | 1.0, 0.17, 0.21 | 1.0, 0.119, 0.23 |
| E (imagesᵣₑₛ) | T₁ | 1 | 1 | 1 |
| | PD | — | 1 | 1 |
| | T₂ | 1 | 1 | 1 |

In general, all contrasts were weighted equally, so that for E (imagesᵣₑₛ) wᵢ^{images}=1, i=1, 2, . . . , N_{contrast}. To compute E (PSFᵣₑₛ), for contrast i and tissue j, the weight wᵢⱼ^{PSF} was chosen to reflect its corresponding magnitude of signal level (k-space data weighting) for the zero phase encoding view denoted by signal (k_{y})ᵢⱼ|ₖ_{_{y}}₌₀.The larger the signal level, the larger the assigned weight wᵢⱼ^{PSF}.

More specifically, normalized weighting factors wᵢⱼ^{PSF} for E(PSFᵣₑₛ) were chosen as:

$${w}_{ij}^{PSF} = \frac{{signal ( {k}_{y} )}_{ij} {\vert}_{{k}_{y} = 0}}{{max}_{j} ( {signal ( {k}_{y} )}_{ij} {\vert}_{{k}_{y} = 0} )} \qquad ( 7 )$$

Finally, for the energy of residue profiles E (profilesᵣₑₛ), normalized weighting factors wᵢⱼ^{profiles} for contrast i and tissue combination j were selected as:

$$\begin{matrix}{w}_{ij}^{profiles} = \frac{α}{{max}_{j} ( α )} \\ α = \frac{1}{{\vert {signal ( {k}_{y} )}_{i , tissue1} \vert}_{{k}_{y} = 0} - {signal ( {k}_{y} )}_{i , tissue2} {\vert}_{{k}_{y} = 0} \vert}\end{matrix} \qquad ( 8 )$$

As expressed in (8), the energy of a residue profile for a specific tissue combination was weighted according to the inverse of the absolute value of the difference in signal level at k_{y}=0 between the two tissues involved. The smaller this difference, the larger the assigned weight wᵢⱼ^{profiles}. This was chosen to weight residue profiles from tissues with similar signal levels, which are converted into similar image intensities after inverse fast Fourier transform (IFFT), more strongly, since deviations from similar intensities might yield less discernible image objects. It should be noted that the choice of weighting factors for the three quantitative criteria described above is not an unique one. Other selections are possible as well.

Referring next to FIG. 16, values for E (PSFᵣₑₛ), E (profilesᵣₑₛ), and E (imagesᵣₑₛ) for optimized 3-contrast fast spin echo combo acquisition protocols and for (non-optimized) combo scans with different parameter variation and/or phase encoding schemes are plotted. For display purposes, residue quantities were scaled to lie within the same range of values. As seen from FIG. 16, optimized combo acquisitions minimized the three selected quantities compared to non-optimized combo scans (see plateaus of small values in all three curves of FIG. 16). The amount of scan time reduction does play a role in selecting a specific protocol as “the optimized combo acquisition”, since residue quantities could be further minimized by reducing the time savings of an acquisition.

The foregoing merely illustrates the principles of the invention. Various modifications and alterations to the described embodiments will be apparent to those skilled in the art in view of the teachings herein. It will thus be appreciated that those skilled in the art will be able to devise numerous systems and methods which, although not explicitly shown or described herein, embody the principles of the invention and are thus within the spirit and scope of the invention.

1. A method for reducing the scan time required to acquire two or more magnetic resonance imaging images of a subject having differing contrasts, comprising the steps of:
(a) selecting a combo acquisition protocol having N sets of parameters, where at least a first set of parameters from said N sets of parameters is different from a second set of parameters from said N sets of parameters;
(b) selecting a set of parameter values from said N sets;
(c) applying at least one RF pulse and at least one gradient field to said subject;
(d) applying a phase encoding gradient field to said subject;
(e) applying a measurement gradient field to said subject simultaneously while measuring an induced signal therefrom;
(f) repeating steps (b) to (e) for each of said N sets of parameters to obtain N measurements; and
(g) processing said N measurements into said two or more images of said subject having differing contrasts by using at least a first portion of said N measurements to reconstruct two or more of said two or more images and a second portion of said N measurements to reconstruct one of said two or more images.

2. The method of claim 1, wherein step (c) includes the steps of applying a first RF pulse and at least a first gradient field to said subject, and then applying a refocusing RF pulse and at least a second gradient field to said subject.

3. The method of claim 2, wherein said first gradient field, said second gradient field, and said measurement gradient field comprise vector fields along one direction.

4. The method of claim 1, wherein said step (a) includes the steps of selecting a number of desired image contrasts, selecting a desired scan time reduction, and optimizing said combo acquisition protocol using said selected number of desired image contrasts and desired scan time reduction.

5. The method of claim 4, wherein said optimizing step includes the steps of selecting a phase encoding scheme and selecting a variation scheme.

6. The method of claim 5, wherein said optimizing step further includes the step of refining said selected phase encoding scheme based on said selected variation scheme.

7. The method of claim 6, wherein said refining step comprises minimizing a measurement of energy of a residue point spread function.

8. The method of claim 6, wherein said refining step comprises minimizing a measurement of energy of a residue profile across any sharp boundaries of said subject.

9 The method of claim 6, wherein said refining step comprises minimizing a measurement of energy of residue images.

10. The method of claim 6, wherein said refining step comprises refining said selected phase encoding scheme so as to preserve contrast between said two or more magnetic resonance imaging images.

11. The method of claim 1, wherein said two or more magnetic resonance imaging images comprises two images, and said step (a) comprises selecting a two contrast spin echo combo acquisition protocol.

12 The method of claim 1, wherein said two or more magnetic resonance imaging images comprises three images, and said step (a) comprises selecting a three contrast spin echo combo acquisition protocol.

13. The method of claim 1, wherein said phase encoding gradient field applied in step (d) is dependent on an index, and further comprising the steps of selecting a unique index value prior to said step (d), applying an inverse phase encoding gradient field prior to step (f), and repeating said steps of selecting a unique index value, applying said index-dependent phase encoding gradient field to said subject; applying a measurement gradient field to said subject simultaneously while measuring an induced signal therefrom, and applying an inverse phase encoding gradient field for each unique index value.

14. The method of claim 13, wherein said two or more magnetic resonance imaging images comprises three images, and said step (a) comprises selecting a three contrast fast spin echo combo acquisition protocol.

15. The method of claim 1, wherein said step (a) comprises selecting a combo acquisition protocol having N unique sets of parameters.

16. A method for reducing the scan time required to acquire two or more magnetic resonance imaging images of a subject having differing contrasts in accordance with a predetermined combo acquisition protocol having N sets of parameters, where at least a first set of parameters from said N sets of parameters is different from a second set of parameters from said N sets of parameters comprising the steps of:
(a) selecting a set of parameter values from said N sets;
(b) applying at least one RF pulse and at least one gradient field to said subject;
(c) applying a phase encoding gradient field to said subject;
(d) applying a measurement gradient field to said subject simultaneously while measuring an induced signal therefrom;
(e) repeating steps (a) to (d) for each of said N sets of parameters to obtain N measurements; and
(f) processing said N measurements into said two or more images of said subject having differing contrasts by using at least a first portion of said N measurements to reconstruct two or more of said two or more images and a second portion of said N measurements to reconstruct one of said two or more images.

17. The method of claim 16, wherein step (b) includes the steps of applying a first RF pulse and at least a first gradient field to said subject, and then applying a refocusing RF pulse and at least a second gradient field to said subject.

18. The method of claim 17, wherein said first gradient field, said second gradient field, and said measurement gradient field comprise vector fields along one direction.

19. The method of claim 16, wherein said phase encoding gradient field applied in step (c) is dependent on an index, and further comprising the steps of selecting a unique index value prior to said step (c), applying an inverse phase encoding gradient field prior to step (e), and repeating said steps of selecting a unique index value, applying said index-dependent phase encoding gradient field to said subject; applying a measurement gradient field to said subject simultaneously while measuring an induced signal therefrom, and applying an inverse phase encoding gradient field for each unique index value.

20. The method of claim 19, wherein said predetermined combo acquisition protocol comprises a protocol having N unique sets of parameters.
